(19) United States
(12) Patent Application Publication
(10) Pub. No.: US 2016/0154513 A1
(43) Pub. Date: Jun. 2, 2016

(54) PORTABLE ELECTRICAL DEVICE CAPABLE OF TESTING BODY FAT PERCENTAGE

(71) Applicant: HON HAI PRECISION INDUSTRY CO., LTD., New Taipei (TW)

(72) Inventors: CHENG-CHING CHIEN, New Taipei (TW); JUN-JIN WEI, New Taipei (TW)

(21) Appl. No.: 14/688,711

(22) Filed: Apr. 16, 2015

(30) Foreign Application Priority Data
Nov. 28, 2014 (CN) 201410712060.6

(51) Int. Cl. G06F 3/041; A61B 5/00

(52) U.S. Cl. CPC G06F 3/0414; A61B 5/4872

(57) ABSTRACT

A portable electrical device includes a shell, a display device, a power management device, a touch button positioned on the shell, and a conductive substrate positioned inside the shell. The conductive substrate is spatially corresponding to the touch button and spaced apart a predetermined distance from the touch button. When a force is applied by a user's finger to push the touch button to touch the conductive substrate, the power management device provides a predetermined subtle alternating current to the user's finger to test a body fat percentage of the user and the display device displays the body fat percentage.

### FIELD

The subject matter herein generally relates to portable electrical devices, and particularly to a portable electrical device capable of testing body fat percentage.

### BACKGROUND

Nowadays, some portable electrical devices have the function that tests the body fat, blood pressure, electrocardiogram, etc. This facilities users of the portable electrical devices.

### BRIEF DESCRIPTION OF THE DRAWINGS

Implementations of the present technology will now be described, by way of example only, with reference to the attached figures, wherein:

FIG. 1 is a block diagram of a system of testing body fat percentage according to an embodiment of the instant disclosure, the system running in a potable electrical device.

FIG. 2 is a diagrammatic view of the potable electrical device of FIG. 1, the potable electrical device including touch buttons.

FIG. 3 shows a diagram of a contact impedance and a force applied by a user to one of the touch buttons.

FIG. 4 is a diagrammatic view of one of the touch buttons.

FIG. 5 is another diagrammatic view of the touch button of FIG. 4, the touch button being pressed.

FIG. 6 is a diagram showing how to test a bioelectrical impedance of the user.

FIG. 7 is a block diagram of the system of testing body fat percentage of FIG. 1.

FIG. 8 is a flowchart illustrating a method of testing body fat percentage according to an embodiment of the instant disclosure.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The term “comprising” means “including, but not necessarily limited to”; it specifically indicates open-ended inclusion or membership in a so-described combination, group, series and the like.

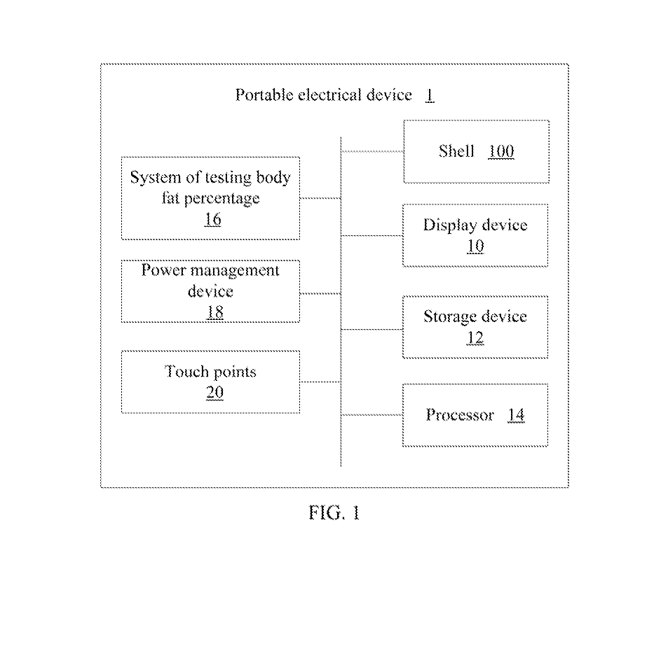

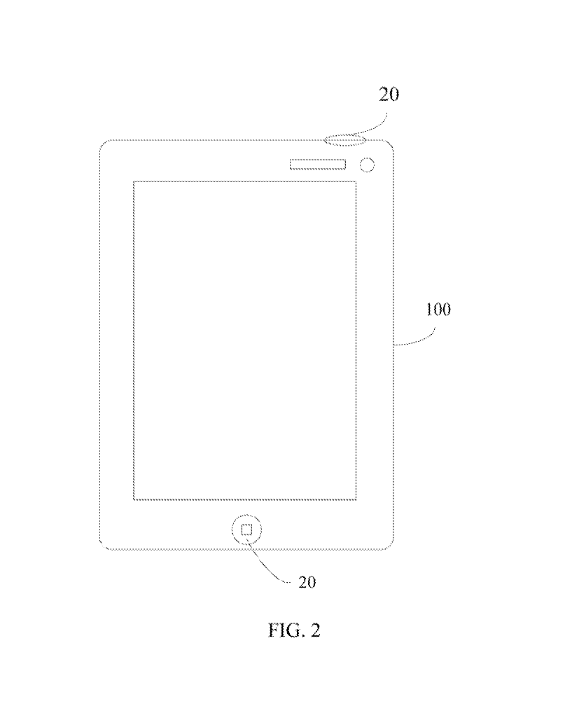

Referring to FIGS. 1 and 2, a portable electrical device 1 for testing body fat percentage is shown. The portable electrical device 1 includes, but not limited to, a shell 100, a display device 10, a storage device 12, a processor 14, a system 16 of testing body fat percentage, a power management device 18, and a plurality of touch buttons 20 electrically coupled to the power management device 18. The portable electrical device 1 can be a smart cell phone, a tablet computer, or a personal digital assistant, etc.

The touch buttons 20 can be positioned on a front surface of the shell 100 and spaced apart from a predetermined distance. In the illustrated embodiment, the portable electrical device includes two of the touch buttons, the two touch buttons 20 are individually positioned on the bottom side of the front surface and on the right upper side of the front surface. The touch buttons 20 are conductive metal buttons and the shell 100 is made of dielectric material.

The display device 10 is for displaying the body fat percentage of a user. In the illustrated embodiment, the display device 10 is a touch panel display.

The storage device 12 is for storing personal data input by the user, for example, height, weight, etc. The storage device 12 can be a RAM of the portable electrical device 1. The storage device 12 can also be a smart media card, a secure digital card, a flash card, or other external storage devices.

The processor 14 controls the system 16 of testing body fat percentage to analyse and process the data input by the user and the tested data.

In the illustrated embodiment, bioelectrical impedance analysis (BIA) is applied. The BIA applies a safe and subtle alternating current to flow through the body of the user to test the body fat percentage of the user. When the alternating current flows in the user's body, electricity is flowing through water which has high conductivity. Impedance of body-composing constituents like water, fat, muscle and so on appears different from one another and the impedance has steady relationship to body composition, therefore body composition can be evaluated using impedance. The BIA uses the two factors that human body is composed of highly conductive tissue (Conductor: Lean body mass) and less conductive tissue (Insulator: Body fat) and measured impedance reflects the ratio between conductive tissue and non-conductive tissue.

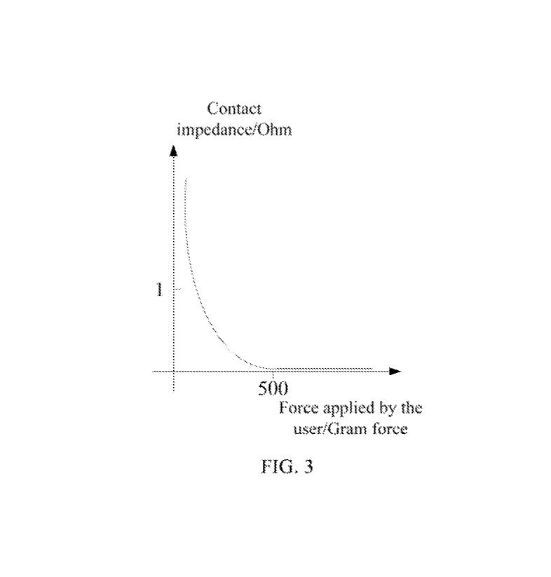

In testing, the alternating current I is provided to flow in the user's body, and the voltage potential U through the user's body is obtained to calculate the impedance Z. The calculated impedance Z includes the bioelectrical impedance Z_{X} and the contact impedance Z₁, Z₂ between the touch buttons 20 and the user, i.e., Z=U/I=Z_{X}+Z₁+Z₂. The contact impedance is changed depending on the contact between the touch buttons 20 and the user's fingers. This affects the accuracy of the test result. FIG. 3 shows the relationship of the contact impedance and the force applied by the fingers to the touch buttons 20. The greater the force is, the less the contact impedance is, and the contact impedance trends to be stable when the force is greater. The alternating current I is equal to or less than 800 milliamperes.

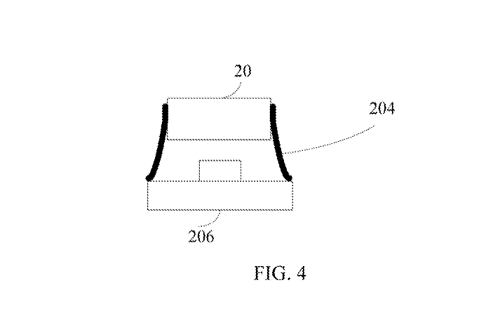

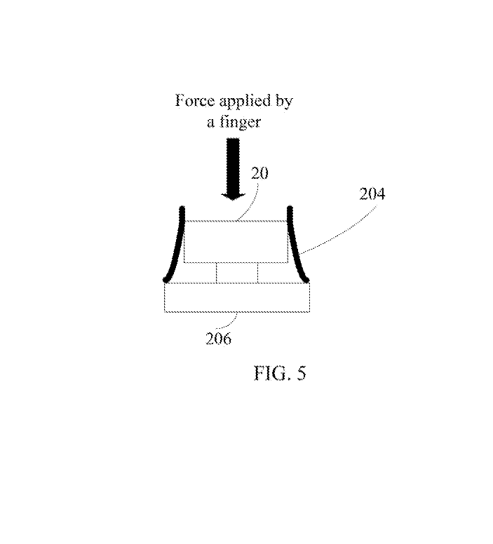

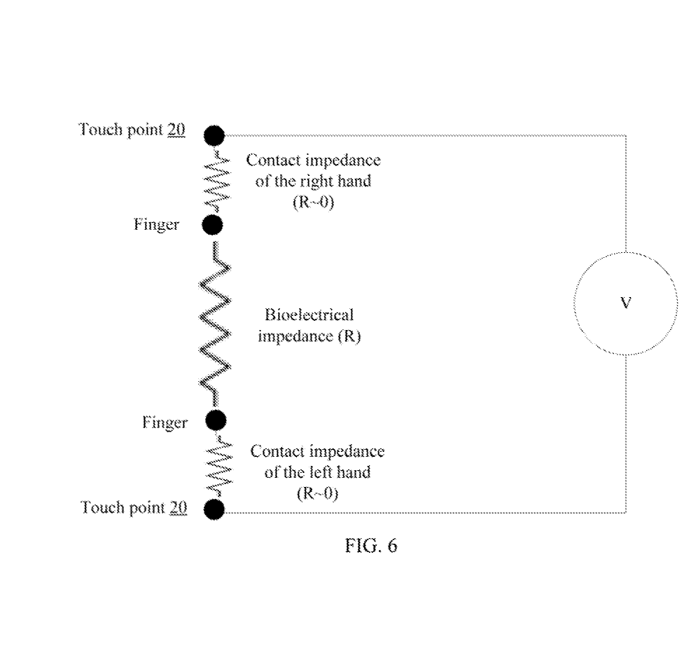

Referring to FIGS. 4, 5, and 6, an embodiment of the touch buttons 20 is shown. The touch buttons 20 are positioned on the shell 100. The portable electrical device 1 includes a plurality of conductive substrates 206 inside the shell 100. Each of the conductive substrates 206 is spatially corresponding to a respective one of the touch buttons 20. The portable electrical device 1 includes two elastic support sheets 204 positioned at two sides of each conductive substrate 206. The elastic support sheets 204 connect to two sides of the touch button 20 and support the touch button 20. When a force is applied by a finger of the user to the touch button 20, the elastic support sheets 204 are compressed and the touch button 20 is moved to contact the conductive substrate 206. In the illustrated embodiment, as shown in FIGS. 5 and 6, the user can use two fingers, for example, the thumb of the left hand and the middle finger of the right hand to press the touch buttons 20. The touch button 20 touches the conductive substrate 206 upon a condition that the force applied by the finger of the user is equal to or more than 500 gram force. The experiments of FIGS. 5 and 6 show that the contact impedance between the touch button 20 and the finger of the user is approximately equal to 0 ohms when the force applied by the finger of the user to the touch button 20 is equal to or more than 500 gram force. When the contact impedance is approximately equal to 0 ohms, the obtained impedance Z is approximately equal to the bioelectrical impedance Z_{X}. Therefore, the tested body fat percentage is almost the actual body fat percentage.

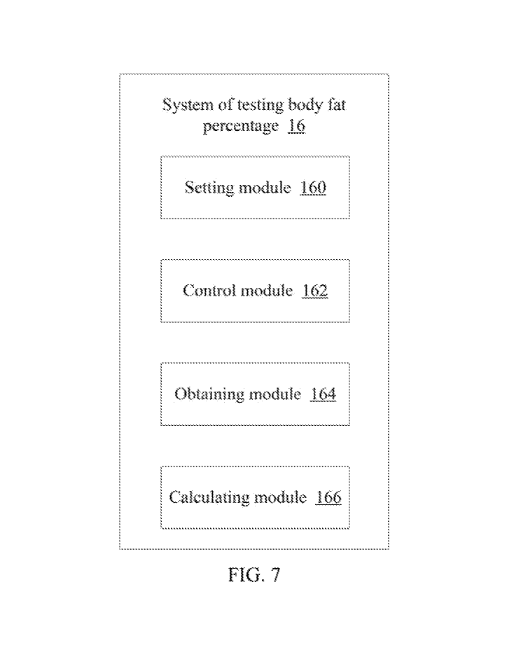

Referring to FIG. 7, an embodiment of the system 16 includes a setting module 160, a control module 162, an obtaining module 164, and a calculating module 166. The word “module,” as used hereinafter, refers to a collection of software instructions which are stored in storage device 12 and can be executed by the processor 14.

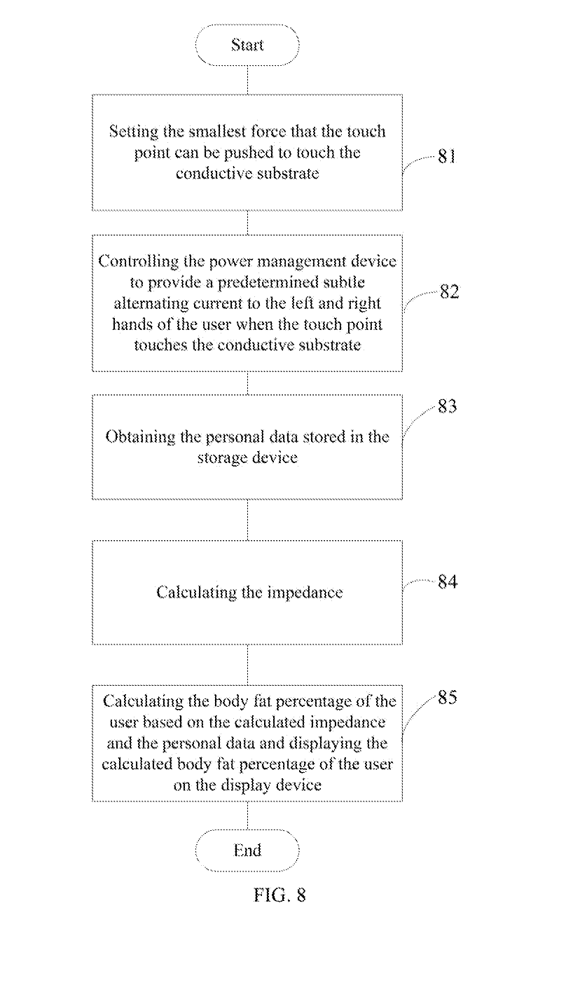

Referring to FIG. 8, a flowchart is presented in accordance with an example embodiment. An example method is provided by way of example, as there are a variety of ways to carry out the method. The example method described below can be carried out using the configurations illustrated in FIG. 8, for example, and various elements of these figures are referenced in explaining example method. Each block shown in FIG. 8 represents one or more processes, methods or subroutines, carried out in the example method. Furthermore, the illustrated order of blocks is illustrative only and the order of the blocks can change according to the present disclosure. Additional blocks can be added or fewer blocks may be utilized, without departing from this disclosure. The example method can begin at block 81.

At block 81, the setting module 160 sets the smallest force that the touch button 20 can be pushed to touch the conductive substrate 206. In the illustrated embodiment, the smallest force is 500 gram force.

At block 82, the control module 162 controls the power management device 18 to provide a predetermined subtle alternating current to the left and right hands of the user when the touch button 20 touches the conductive substrate 206. The subtle alternating current is equal to or less than an equal to 800 milliamperes. In the illustrated embodiment, the power management device 18 provides the subtle alternating current to the user upon a condition that the fingers of the users apply a force equal to or more than 500 gram force to the touch buttons 20 to push the touch buttons 20 to touch the conductive substrate 206.

At block 83, the obtaining module 164 obtains the personal data stored in the storage device 12. The personal data can be height, gender, weight, etc.

At block 84, the calculating module 166 calculates the impedance. The calculating module 166 obtains the voltage potential through the right and left hands and calculates the impedance based on the obtained voltage potential and the predetermined subtle alternating current.

At block 85, the calculating module 166 calculates the body fat percentage of the user based on the calculated impedance and the personal data and displays the calculated body fat percentage of the user on the display device 10.

In other embodiments, at block 83, if the storage device 12 does not have the personal data, the system 16 reminds the user to input the personal data. The obtaining module 164 obtains the personal data after the user inputs the personal data into the storage device 12.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A portable electrical device, comprising:
a shell;
a touch button positioned on the shell;
a conductive substrate positioned inside the shell spatially corresponding to the touch button and spaced apart a predetermined distance from the touch button;
a power management device configured to provide, when a force is applied by a user's finger to push the touch button to touch the conductive substrate, a predetermined subtle alternating current to the user's finger to test a body fat percentage of the user; and
a display device configured to display the body fat percentage.

2. The portable electrical device of claim 1, comprising two elastic support sheets positioned at two sides of the conductive substrate, and the two elastic support sheets connecting to two sides of the touch button and supporting the touch button.

3. The portable electrical device of claim 1, wherein the force applied by the user's finger is equal to or more than 500 gram force.

4. The portable electrical device of claim 1, wherein the predetermined subtle current is equal to or less than 800 milliamperes.

5. The portable electrical device of claim 1, comprising:
a storage device;
a processor; and
one or more programs that are stored in the storage device and are executed by the processor, the one or more programs comprising:
a setting module that sets the smallest force that the touch button is pushed to touch the conductive substrate;
a control module that controls the power management device to provide the predetermined subtle alternating current to the user's finger when the touch button touches the conductive substrate;
an obtaining module that obtains a personal data of the user; and
a calculating module calculates an impedance and calculates the body fat percentage of the user based on the calculated impedance and the personal data.

6. The portable electrical device of claim 5, wherein the calculating module obtains the voltage potential through the user's body and calculates the impedance based on the obtained voltage potential and the predetermined subtle alternating current.

7. The portable electrical device of claim 5, wherein the calculating module displays the calculated body fat percentage of the user on the display device.

8. The portable electrical device of claim 5, wherein the personal data is input by the user.

9. The portable electrical device of claim 5, wherein the personal data is stored in the storage device.

10. A method of testing body fat percentage of a user, comprising:
providing a portable electrical device which comprises:
a shell;
a display device;
a power management device;
a touch button positioned on the shell; and
a conductive substrate positioned inside the shell, the conductive substrate spatially corresponding to the touch button and spaced apart a predetermined distance from the touch button;
setting the smallest force that the touch button is pushed to touch the conductive substrate;
controlling the power management device to provide a predetermined subtle alternating current to the user's finger when the touch button touches the conductive substrate;
obtaining the personal data of the user;
calculating an impedance; and
calculating the body fat percentage of the user based on the calculated impedance and the personal data.

11. The method of claim 10, wherein the smallest force is equal to or more than 500 gram force.

12. The method of claim 10, wherein the predetermined subtle current is equal to or less than 800 milliamperes.

13. The method claim 10, wherein the impedance is calculated based on the voltage potential through the user's body and the predetermined subtle alternating current.

14. The method claim 10, comprising:
displaying the calculated body fat percentage of the user on the display device.

15. The method claim 10, wherein the personal data is input by the user.

16. The method claim 10, wherein the portable electrical device comprises a storage device, and the personal data is stored in the storage device.
